(19) United States
(12) Patent Application Publication
(10) Pub. No.: US 2016/0192682 A1
(43) Pub. Date: Jul. 7, 2016

(54) FORMULATION AND PROCESS FOR MAKING FERMENTED PROBIOTIC FOOD AND BEVERAGE PRODUCTS

(71) Applicant: UNIVERSITY OF CENTRAL OKLAHOMA, Edmond, OK (US)

(72) Inventors: Kanika Bhargava, Edmond, OK (US); Carissa Jetto, Edmond, OK (US); Maurice Haff, Edmond, OK (US)

(21) Appl. No.: 14/982,332

(22) Filed: Dec. 29, 2015

Related U.S. Application Data
(60) Provisional application No. 62/098,816, filed on Dec. 31, 2014.

(51) Int. Cl. A23L 1/20; C12N 1/20; A23C 11/10

(52) U.S. Cl. CPC A23L 1/2008; C12N 1/20; A23L 1/3014; A23C 11/106; A23K 1/009; A23L 1/211; A23K 1/007; A23V 2002/00; A23Y 2220/15; A23Y 2240/75

(57) ABSTRACT

A process providing a method for production of probiotic functional food products from plant substrates, the method comprising: activation of probiotic bacteria in an agar-agar formulation; formulation of water active plant substrates; culturing and incubation of water active plant substrates with activated probiotic bacteria; cooling and refrigeration.

### CROSS REFERENCES TO RELATED APPLICATIONS

This application claims the benefit of U.S. Provisional Patent Application No. 62/098,816 filed on Dec. 31, 2014 in the name of Kanika Bhargava and Carissa Jetto, which is expressly incorporated herein by reference in its entirety.

### FIELD OF THE INVENTION

The present invention relates to a process for making fermented probiotic products. More particularly, the invention relates to a process and formulation for making, fermented probiotic food and beverage products from starter cultures activated in agar-agar that substantially exhibit characteristics of traditional artisan products.

### BACKGROUND

Growing consumer awareness regarding gut health has pushed the demand for probiotic products including food, beverages, and dietary supplements. Food and beverages have accounted for the greatest demand for probiotic products. The World Health Organization defines probiotics as “live microorganisms which, when administered in adequate amounts, confer a health benefit on the host.” Products such as fermented meat, dairy, bakery, grains, fats, oils, beverages, fish, eggs, vegetables, fruit, and legumes can contain these live microorganisms. Probiotics are microflora proven safe to consume in fermented foods such as fruit and vegetable juices, yogurts, and pickled edibles. Further, probiotics are being used as food additives and in supplements. The bacterial genera Lactobacillus and Bifidobacterium constitute the most frequently employed probiotics in preparations for human use, and are often used in fermentation of animal feed stocks. Probiotic products are regulated by the U.S. Food and Drug Administration (FDA). Regulations promulgated by the FDA govern manufacturer responsibilities, labeling and safety of these products, whether in food, supplement, or drug form.

Lactic acid bacteria (LAB) are considered the most important bacteria in desirable food fermentations producing probiotic products. Lactic acid bacteria are a group of Gram positive bacteria, non-respiring, non-spore forming, cocci or rods, which produce lactic acid as the major end product of the fermentation of carbohydrates. Microbiologists use gram staining techniques to classify bacteria into two groups: gram-positive or gram-negative. The positive/negative reference relates to the bacterium's chemical and physical cell wall properties. Most microorganisms recognized to date as probiotics are Gram-positive. Microflora from the genera Lactobacillus, Leuconostoc, Pediococcus and Streptococcus are the main LAB species involved in food products, however, a plurality of other species have been identified, but may play a lesser role in lactic fermentations,

Lactic acid bacteria (LAB) are microaerophilic that means they grow well under conditions of low oxygen content. They convert carbohydrates such as lactose to lactic acid plus carbon dioxide and other organic acids. As a result of the microaerophilic characteristic, lactic acid bacteria do not cause drastic changes in food composition. Some LAB are homofermentative, producing only lactic acid; others are heterofermentative and produce lactic acid plus other volatile compounds and small amounts of alcohol. Examples of lactic acid-producing bacteria involved in food fermentations include Lactobacillus acidophilus, L. bulgaricus, L. plantarum, L. caret, L. pentoaceticus, L brevis and L. thermophilus.

Homofermenter L. plantarum produces high acidity in all plant (e.g., vegetable, legume) fermentations and plays a primary role. All tactic acid producers are non-motile gram positive rods that need complex carbohydrate substrates as a source of energy. The lactic acid produced is effective in inhibiting the growth of other bacteria that may decompose or spoil the food. Lactic acid bacteria produce specific reactions and exhibit a diverse metabolic capacity that makes them very adaptable to a range of conditions largely responsible for acid food fermentations.

Microflora vary in their optimal pH requirements for growth. Most bacteria favor conditions with a near neutral pH (7). However, certain bacteria are acid tolerant and will survive as pH levels are reduced. Notable acid-tolerant bacteria include the Lactobacillus and Streptococcus species, which are microaerophilic and are used extensively in the fermentation of dairy and vegetable products.

Different bacteria can tolerate different temperatures across a range of fermentations. Most bacteria have a temperature optimum of between 20 to 30 degrees Centigrade (° C.). Some (the thermophiles) prefer higher temperatures (50 to 55″C), while other bacteria exhibit colder temperature optima (15 to 20° C.). Most lactic acid bacteria produce desired results at temperatures in the range of 18 to 22° C. The Leuconostoc species which initiate fermentation have an optimum temperature range between 18 to 22° C. Temperatures above 22° C., favor the lactobacillus species.

Lactic acid bacteria (LAB) exhibit tolerance to high salt concentrations. High salt concentrations in diy or brine form are used extensively in fermenting vegetables to draw out juices to promote fermentation. The salt tolerance of LAB gives them an advantage over other less tolerant species and allows the lactic acid fermenters to begin metabolism, producing an acidic environment further inhibiting growth of non-desirable organisms. Leuconostoc is noted for its high salt tolerance and for this reason, initiates the majority of lactic acid fermentations where salt is used.

In general, bacteria require a fairly high water activity (0.9 or higher) to survive. There are a few species that tolerate water activities lower than this, but usually the yeasts and fungi will predominate on foods with a lower water activity. The term “water activity” refers to water in food which is not bound to food molecules. This unbound water can support the growth of bacteria, yeasts and molds (fungi). For a food to have a useful shelf life without relying on refrigerated storage, it is necessary to control either its acidity level (pH) through fermenting or the level of water activity through drying or a suitable combination of the two.

Lactic acid fermentations are carried out under three basic types of condition: dry salted, brined, and non-salted. Salting provides a suitable environment for lactic acid bacteria (LAB) to grow. Sauerkraut is one example of an acid fermentation of vegetables. The ‘sauerkraut process’ shown in FIG. 1 can be applied to any other suitable type of vegetable product. Shredded cabbage or other vegetables exhibiting high water content are placed in a container and salt is added. Salting along with mechanical pressure applied to the cabbage or vegetables expel the juice, which contains fermentable sugars and other nutrients suitable for microbial activity.

The use of salt brines is common in fermenti vegetables that have low water content, but not typically used in making sauerkraut. As shown in FIG. 2, vegetables are submerged in brine, ensuring that none float on the surface to avoid spoilage. The strong brine draws the sugar and water out of the vegetables, and simultaneously reduces the salinity of the solution. In order to maintain a salt solution that supports fermentation, more salt must be added to the brine solution. If the concentration of salt falls below 12%, it may result in spoilage of the vegetables.

A few vegetables may be fermented by lactic acid bacteria (LAB), without the prior addition of salt or brine. Non-salted food products include gundritk (consumed in Nepal), sinki and other wilted fermented leaves. Fermentation of animal feed stocks (e.g. seed grains) without salt is often used to reduce anti-nutrient properties of seed grains, making the feed stocks more available for digestion. However, yeast can predominate producing alcohol in fermentation at low temperatures typical of farm environments, Fermentation of vegetables for human consumption without the use of salt or brine produces a low-sodium product. However, the fermentation process without added salt or brine requires rapid colonization of the food by lactic acid producing bacteria. Absent a high rate of colonization, the pH level will not decrease fast enough to produce an environment unsuitable for the growth of spoilage organisms, including bacteria and yeast. Oxygen must also be excluded to favor growth of Lactobacilli and prevent growth of yeasts. These factors can pose significant challenges in producing fermented products at scale without using salt or brine.

In order to produce fermented foods of consistent quality, starter cultures such as those used in the dairy industry have been recommended. Starter cultures comprise specific bacteria that serve to ensure greater consistency between fermentation batches and speed up the fermentation process eliminating or reducing the time lag while the relevant microflora colonize a food substrate. Because the starter cultures used are acidic, they also inhibit the undesirable micro-organisms. Because these organisms only survive Dora short time (long enough to initiate the acidification process), they do not disturb the natural sequence of micro-organisms. An example of the use of a starter culture in producing a fermented food product from flax seed without adding salt is disclosed in European Patent EP2003986 A1. A suspension of defatted crushed flaxseed is fermented by a starter culture which comprises probiotic bacteria, and seasoned and stabilized, to produce a spoonable or drinkable fermented snack product.

Fermentations that rely on dry-salting (FIG. 1) or the use of brines (FIG. 2) produce finished products that suffer high sodium content, which is considered detrimental to human health. U se of starter cultures can reduce or eliminate the need for salt in fermentation processes, however, accelerated growth of microflora to colonize the food substrate is essential to success of fermentation without the prior addition of salt. Absent rapid colonization by desirable microflora, fermentation may be incomplete and the food substrate may spoil resulting in a failed production process. Assuring rapid colonization without the use of salt becomes increasingly difficult as batch size increases. An effective method to accelerate microflora growth is needed to consistently produce low sodium fermented food products at commercial scale.

Yogurt and various probiotic preparations have developed into a well-accepted and consumed class of fermented dairy products. Traditional yogurt is a fermented product made using milk produced by animals (e.g., cows, goats, and sheep). Traditional kefir is a fermented milk product produced using a combination of yeasts and probiotic bacteria. However, demand for yogurt-style products made from non-dairy food sources (e.g. soy, almonds, coconut) is increasing rapidly, reportedly due to lactose intolerance in adults and increased incidence of food allergies in both adults and children.

The National Institutes of Allergy and Infectious Diseases (NIAID) reported that approximately 5 percent of children and 4 percent of adults in the United States suffer food allergies. Tree nut allergy is one of the most common food allergies in children and adults. Tree nuts can cause a severe, potentially fatal, allergic reaction (anaphylaxis). An allergy to tree nuts tends to be lifelong; recent studies have shown that only about 9 percent of children with a tree nut allergy eventually outgrow their allergy. Approximately 0.4 percent of children are allergic to soy. Studies indicate that an allergy to soy generally occurs early in childhood and ollen is outgrown by age three. Research indicates that the majority of children with soy allergy will outgrow the allergy by the age of 10. Approximately 2.5 percent of children younger than three years of age are allergic to milk. Nearly all infants who develop an allergy to milk do so in their first year of life, however, most children eventually outgrow a milk allergy.

Milk allergy should not be confused with lactose intolerance. A food allergy is an overreaction of the immune system to a specific food protein. When the food protein is ingested, in can trigger an allergic reaction that may include a range of symptoms from mild symptoms (rashes, hives, itching, swelling, etc.) to severe symptoms trouble breathing, wheezing, loss of consciousness, etc.). A food allergy can be potentially fatal. Unlike food allergies, food intolerances do not involve the immune system. People who are lactose intolerant are missing the enzyme lactase, which breaks down lactose, a sugar found in milk and dairy products. As a result, lactose-intolerant patients are unable to digest these foods, and may experience symptoms such as nausea, cramps, gas, bloating and diarrhea. While lactose intolerance can cause great discomfort, it is not generally life-threatening.

Food allergy among children in the United States rose 18 percent from 1997 to 2007, according to the Centers for Disease Control and Prevention. Reasons for this increase remain unclear, but recent studies have suggested that environmental factors play an important role by changing the composition of the commensal bacteria that colonize the intestinal tract. These trillions of bacteria, collectively known as the intestinal microbiota, are vital for health and immune system development. The rise in antibiotic use during childhood has been linked to an increased risk of allergic diseases, suggesting that in addition to destroying infectious bacteria, these drugs also can alter the composition of the microbiota. Researchers have found that certain naturally occurring gut bacteria may protect against food allergen sensitization—a key step in the development of food allergy. Yogurt-style products can be a source for restoring healthy gut bacteria.

Yogurt is usually biologically acidified by means of adding Lactobacillus bulgaricus and Streptococcus thermophilus, the pH of which are about 4.1 to 4.6. Typical yogurt products have a gelled texture of varying density depending on the process used in production. The texture may also be creamy or liquid, Exemplary tbrms of yogurt and related products may be a gel-like form (e.g. “Greek” yogurt), a stirred yogurt (e.g., including fruit mixtures), or a drinking yogurt in a liquid form (e.g., similar to kefir). The difference between Greek and regular yogurt occurs after fermentation. Greek yogurt goes through a straining process that removes most of the whey, resulting in a thicker form of yogurt. Another essential difference between Greek yogurt and regular yogurt is that Greek yogurt contains both cream and milk while regular yogurt contains only milk. Both kefir and yogurt are cultured (i.e. fermented) milk products, but they contain different types of beneficial bacteria. Yogurt contains transient beneficial bacteria that keep the digestive system clean and provide food for the preferred bacteria that may reside there. In contrast, kefir contains bacteria that form colonies in the intestinal tract and may remain there, unlike the bacteria in yogurt which is more transient.

Kefir is a fermented milk drink made with kefir “grains” (a yeast/bacterial fermentation starter). It is prepared by inoculating cow, goat, or sheep milk with kefir grains. Kefir grains are a combination of lactic acid bacteria and yeasts in a matrix of proteins, lipids, and sugars, and this symbiotic matrix forms “grains” that resemble cauliflower. For this reason, a complex and highly variable community of lactic acid bacteria and yeasts can be found in these grains although some predominate; Lactobacillus species are always present. Several varieties of probiotic bacteria are found in kefir products not commonly found in yogurt; these may include Lactobacillus acidophilus, Bilidobacterium bilidum, Streptococcus thermophilas, Lactobacillus delbrueckii subsp. bulgaricus, Lactobacillus helveticus, Lactobacillus kefiranotaciens, Lactococcus lactis, and Lenconostoc species. Kefir also contains beneficial. yeasts, such as Saccharomyces kefir and Torula kefir, which dominate, control and eliminate destructive pathogenic yeasts in the body.

Kefir grains can ferment milk obtained from most mammals, and offers capabilities to continue to grow in such milk matrix. Raw milk has been traditionally used. Kefir grains can also ferment milk substitutes such as soy milk, rice milk, and coconut milk, as well as other sugary liquids including fruit juice, coconut water, and ginger beer. However, the kefir grains may cease growing if the medium used does not contain all the growth factors required by the bacteria.

As recited in U.S. Pat. No. 6,399,122B2, the basic yogurt manufacturing processes generally uses a dairy medium such as milk or a milk component as starting material in a manufacturing process as depicted in FIG. 3. The dairy medium is typically chosen from, but is not limited to, pasteurized or unpasteurized milk, cream, non-fat dried milk or concentrated milk and water. Other ingredients, such as various thickening agents/stabilizers hydrocolloids such as starches or gelatins), and/or whey protein concentrates can optionally be added to adjust gel structure and/or consistency and the mixture is then heated to allow pasteurization and thickening. To this mixture is added yogurt-producing bacterial culture(s), and fermenting proceeds under heated conditions until the mixture reaches the required level of acidity to produce the yogurt. Fruit, flavorings, or colorants can optionally be added to the yogurt to produce the final commercial product.

With respect to the dairy medium used in typical yogurt producing processes, certain percentages of fat and dry matter are chosen depending upon the final product desired. In order to obtain the desired gel structure in the yogurt with the desired consistency, the natural tiontht dry matter content can be adjusted by either addition of dry matter or by proper selection of the dairy medium starting material. For example, low-fat or skim milk yogurt has a softer gel than a whole milk yogurt; therefore, the matter content can be raised by addition of dry matter such as milk concentrate or milk powder or by water removal through evaporation.

Typically, optional ingredients are added to the dairy medium to adjust gel properties. For example, a typical process would use a starting mixture containing whey protein concentrate in the range of 0 to about 2%, a starch component in the range of 0 to about 5%, a sweetener in the range of 0 about 20%, a gelatin component in the range of 0 to about 3%, with the remainder of the mixture being a dairy medium (e.g., milk or milk componen(s) or a non-dairy medium such as soy, almonds, or coconut.

The mixture is generally pasteurized. This process deactivates spoilage causing micro-organisms. This pasteurization and thickening is generally accomplished by heating the mixture to about 180° F. to about 200° F. for about 2 to about 12 minutes, typically about 6 to about 9 minutes. After this heating step, the mixture is typically allowed to cool to about 105° F. to about 110° F. and placed into a fermentation tank wherein the temperature is continually maintained within the range of about 105° F. to about 110° F., yogurt culture is added. Fermentation takes place until the mixture reaches appropriate levels of acidity. Acidification causes the coagulation of proteins that are responsible for the typical yogurt texture. The typical yogurt flavor develops during acidification.

Starter cultures for yogurt generally are thennophilic (heat-loving) bacteria. Typical yogurt cultures are Streptococcus thermophilus and Lactobacillus bulgaricus. These bacteria are used in yogurt production because they can thrive and produce lactic acid at the temperatures used in conventional yogurt manufacturing. In the typical yogurt production process, fermentation proceeds until the pH of the mixture is below approximately 4.6. Below a pH of about 4.6 the final product is considered a high acid food and the product will not support growth of pathogenic bacteria. The fermentation step is usually between 2 and 12 hours, more typically between 2 and 4 hours.

In typical yogurt producing processes, after the fermentation process has passed and the pH level has reached approximately 4.6, the mixture is cooled to about 35° F. to about 45° F., typically about 40° F, resulting in the final yogurt product. The yogurt is sent to a storage tank, and from the storage tank the yogurt is sent to be packaged for sale. Other components, such as fruit, flavoring, coloring or sweetener can optionally be added previous to storage, during storage, or between storage and packaging.

As recited in U.S. patent application Ser. No. 13/008,132 the conventional yogurt making process often requires double pasteurization of the product. In the conventional process, the milk is pasteurized first to deactivate and kill the milk borne naturally occurring bacteria. Yogurt can be further pasteurized after incubation to kill and deactivate the live bacterial culture before serving. After incubation, the milk is converted to yogurt and then the yogurt is pasteurized again for deactivating and killing the active cultures prior to storage at 4° C. Repeated pasteurization processes reduce the nutritional value of milk as well as consume significant amounts of energy to heat and cool the yogurt for short periods of time. Although the pasteurization process does not kill all the bacteria present in the culture, it significantly reduces the live cell number in the yogurt. After pasteurization some of the residual live bacterial organisms which came from the active cultures stay alive in the yogurt. These residual organisms reduce the shelf life of yogurt and also increase the chances of contamination if the container is left open and not consumed completely when it is opened for consumption.

A process now typical for producing non-dairy yogurt is recited in U.S. Pat. No. 3,950,544 and illustrated in FIG. 4. According to the process disclosed, a non-dairy yogurt can be prepared by leaching soybean meal with an aqueous solution having a pH of 4 to 5 to remove sugars without removing protein, and then leaching a resultant residual sugar-free cake with an aqueous solution having a pH above 7 to dissolve protein material. The solution is strained to remove residual solids. The pH of a resulting protein-containing filtrate may then be adjusted to 6.5 to 7.0, and sugar added to the filtrate and homogenizing to produce a soymilk. The sugar used in the homogenization step is needed to enable fermentation. The type of sugar is selected to be compatible for utilization by the bacteria used in the inoculation step. The soy milk is sterilized at about 116° C., and fermented with a lactic culture to produce a non-dairy yogurt. Colorant, flavorings, and fruit mixtures can be added.

Yogurt is sold in supermarkets under refrigerated conditions. Most yogurt available in the markets contain added artificial flavor, fruit, puree, juices and several other additives to maintain the consistency of the product. Several types of chemicals may be added to increase the shelf life of yogurt. Post pasteurized products are also refrigerated to less than 4° C. to enhance the shelf life. Chemicals like sodium phosphate, sorbitol, glycerine and other additives are commonly used to make thicker consistency and longer shelf life. Some of these additives may have animal source as origin of the compound like gelatins. Due to the presence of products of animal origin, vegetarians and others either by religious practice or lifestyle choice do not consume the products. Further, with the increase in allergies to nuts, soy, and dairy the population is in need of a fermented non-dairy alternative that provides consumers with higher nutrients, fiber and protein, with greatly diminished risk of allergic reaction and digestive intolerance.

### SUMMARY OF THE INVENTION

The present invention provides a process for making non-dairy fermented probiotic food products which resemble traditional artisan products. The texture and consistency of the products can be controlled to produce relatively firm or more liquid characteristics depending on the fermented product being produced. The process of the present invention is important because it provides a method to manufacture nutrient dense functional foods that are low in cost to produce, exhibit consistent qualities, and can be manufactured at any scale all over the world. Fermented formulations my include, but are not limited to bakery, grains, fats, oils, beverages, vegetables, fruit, and legumes. The starter culture composition of the present invention may also be used in processes adapted to produce all types of fermented and probiotic food products for humans, as well as fermented and probiotic feed stocks for animals.

In a broad aspect, the present invent o provides formulation and process for making fermented and probiotic products from a plurality of plant substrates without the use of salt by increasing the water activity of the substrate and accelerating beneficial microflora colonization through addition of a starter culture comprising at least one species of microflora activated in agar-agar. Agar-agar is derived from the polysaccharide agarose, which forms the supporting structure in the cell walls of certain species of algae, and which is released on boiling. Agar-agar is actually the resulting mixture of two components: the linear polysaccharidk. agarose, and a heterogeneous mixture of smaller molecules called agaropectin. Historically, agar-agar has been chiefly used as an ingredient in desserts throughout Asia and also as a solid substrate to contain culture media for microbiological laboratory work. Agar-agar can be used as a vegetarian substitute for gelatin, a thickener for soups, in fruit preserves, ice cream, and other desserts. For commercial purposes, it is derived primarily from Gelidium amansii.:In chemical terms, agar-agar is a polymer made up of subunits of the sugar galactose.

In anotheraspect, substrate formulation and composition is optimized able product characteristics closer to traditional artisan flavors and consistencies.

In another aspect, the process of the present invention may be used to produce a fermented food product from plant sUbstrates comprising vertables,

In another aspect, the process of the present invention may be used to produce a fermented food product from plant substrates comprising fruit.

In another aspect, the process of the present invention may be used to produce a fermented food product from plant substrates comprising grains.

In another aspect, the process of the present invention may be used to produce a fermented food product from plant substrates comprising legumes.

In another aspect, the process of the present invention may be used to produce a non-dairy product from a legume substrate comprising lentils that exhibits substantially the characteristics of traditional dairy-based yogurt.

In another aspect, the process of the present invention may be used to produce a non-dairy product from a legume substrate comprising lentils that exhibits substantially the characteristics of traditional dairy-based kefir.

In another aspect, the ingredients in the formulation of the non-dairy product produced by the process of the present invention have low to no known allergies when produced using lentils, which is an essential characteristic of the yogurt product and the kefir product.

In another aspect, the formulation and process of the present invention may be used to produce a non-dairy yogurt-like product that provides probiotics that are essential thr health of the human gut and digestion.

In another aspect, the formulation and process of the present invention may be used to produce a non-dairy kefir-like product containing probiotics essential for health of the human gut and digestion.

In another broad aspect, the formulation and process of the present invention provides a method to manufacture probiotic functional foods from plant substrates, the method comprising:
a. Activation of probiotic bacteria in agar-agar to produce a starter culture composition;
b. Formulation of plant (e.g., vegetable, grain, fruit or legume) substrates assuring elevated water activity;
c. Culturing and incubation of water active substrates with activated probiotic bacteria in an agar-agar base composition;
d. Cooling and refrigeration of the incubated product.

In another aspect, the formulation and process of the present invention produces a starter culture composition tbr food fermentation comprising at least one species of microllora activated and allowed to grow in an agar-agar base formulation.

In another aspect, the formulation and process of the present inventio produces a fermented functional food product without addition of salt.

In another aspect, the starter culture composition produced using the formulation and process of the present invention may be utilized in a liquid or a more solidified state.

In another aspect, the starter culture composition produced using the formulation and process of the present invention may exhibit a gelled consistency molded into specific shapes.

In another aspect, the starter culture composition produced using the formulation and process of the present invention may be added to any water active vegetable, grain, fruit or legume substrate exhibiting liquid or more solid characteristics to produce a probiotic functional food.

In another aspect, the star er culture composition produced using the formulation and process of the present invention may be added to water to produce a probiotic functional beverage, where the starter culture composition may or may not include flavorings or nutritional additives.

In another broad aspect, the formulation and process of the present invLntion provides a method to manufacture lentil based probiotic yogurt or kk.Tir as a functional food, the method comprising:

a. Activation of probiotic bacteria in agar-agar to produce a starter culture composition;

b. Formulation of lentil milk substrate by increasing water activity of ground lentils;

c. Culturing and incubation of lentil milk with activated probiotic bacteria;

d. Cooling and Refrigeration.

In another aspect, the formulation and process of the present invention may exclude straining to retain substantially all of the fiber and protein present in the lentils.

In another aspect, the formulation and process of the present invention produces a non-dairy yogurt-like product or kefir-like product depending on substrate composition and the specific microflora activated in agar-agar to produce the starter culture composition, where the product produced exhibits a desired texture without the use of gums or thickeners.

In another aspect, the formulation and process of the present invention utilizes prebiotics present in lentils to provide an ideal matrix for probiotic growth and produce a nutrient dense probiotic food product.

### BRIEF DESCRIPTION OF THE DRAWINGS

FIG. 1 depicts a typical process for producing a fermented plant product using dry salt.

FIG. 2 depicts a typical process for producing a fermented plant product using brine.

FIG. 3 depicts a typical dairy-based yogurt manufacturing process.

FIG. 4 depicts atypical non-dairy-based yogurt manufacturing process.

FIG. 5 presents the steps in the process of the present invention producing a fermented plant product using beneficial microflora activated in an agar-agar fommiation,

FIG. 6 depicts the process of the present invention providing a method of producing a fermented product derived from plants (e.g., vegetables, grains, fruits using beneficial microflora activated in an agar-agar formulation.

FIG. 7 presents the steps in the process of the present inven ion producing non-dairy-based yogurt or kefir.

FIG. 8 depicts the process of the present invention providing a method of producing a yogurt-like product derived from lentils and microflora activated in agar-agar formulation.

### DETAILED DESCRIPTION OF THE INVENTION

In brief: The method in accordance with the process of the present invention provides several advantages over previous practices in this field. Unlike the present invention, lactic acid fermentations of plant substrates currently use one of three methods: dry salted, brined, and non-salted. Salting, whether in the form of dry salt or brine, can provide a suitable environment for lactic acid bacteria to grow, but results in a fermented product exhibiting high sodium content. Only a few types of vegetables can be fermented by tactic acid bacteria in an anaerobic atmosphere without the prior addition of salt or brine, rely on uncertain colonization, and produce relatively low-volume batches. Seed grains fermented without salt or brine for animal feed stocks may spoil in low temperature environments typical of farms.

Fermentation of plant substrates without the use of salt becomes increasingly difficult as batch size increases, and may fail because of insufficient colonization of the food product by beneficial micro-flora. The formulations and process provided by the present invention produces a fermented functional food product without addition of salt. This is accomplished by elevating water activity in a food substrate, followed by inoculation with a starter culture composition comprising lactic acid bacteria activated in an agar-agar formulation prior to mixing with the food substrate. Activating and increasing lactic acid bacteria in agar-agar to produce a starter culture composition then mixed with the food substrate assures rapid colonization. The present invention provides an effective method to accelerate growth of beneficial microftora during plant substrate fermentation absent addition of salt or brine to consistently produce low sodium, fermented food products or animal feed stocks at commercial scale.

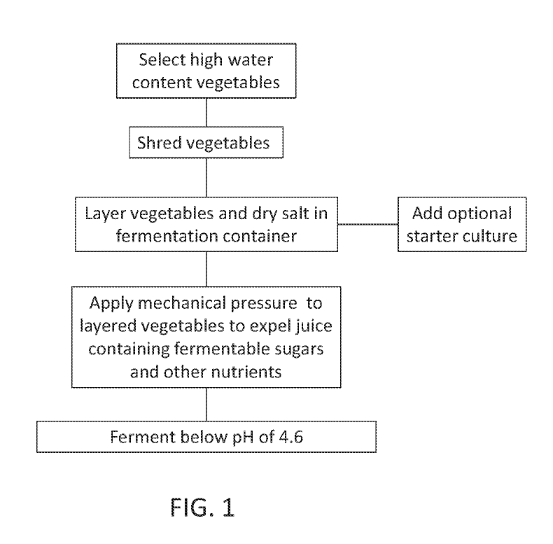

FIG. 1 shows a typical process for acid fermentation of vegetables using dry salt and mechanical pressure applied to shredded vegetables to expel juice. The juice must be expelled to provide fermentable sugars and other nutrients required for microbial activity. Shredded cabbage or other vegetables exhibiting high water content are placed in a container and d. added unlike the method provided by the present invention. Salting along with mechanical pressure applied to the shredded cabbage or vegetables expel the juice, creating an environment needed for microbial activity.

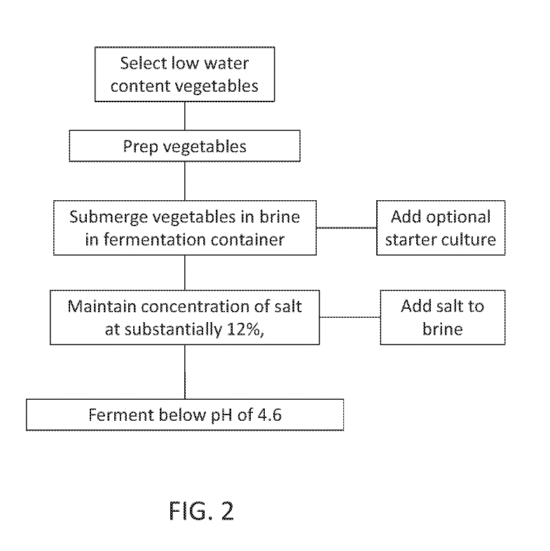

FIG. 2 shows a typical process for acid fermentation of vegetables that have low water activity using salt brines. Unlike the method provided by the process of the present invention, vegetables must be submerged in brine to draw sugar and water out of the vegetables to support microbial activity. This simultaneously reduces the salinity of the solution, so more salt must be added to the brine solution to maintain a salt level above 12% as needed to support fermentation and avoid spoilage of the vegetables through putrefaction and softening. Fermented food products produced using brine exhibit high sodium content. The method provided by the process of the present invention does not require the use of salt or brine.

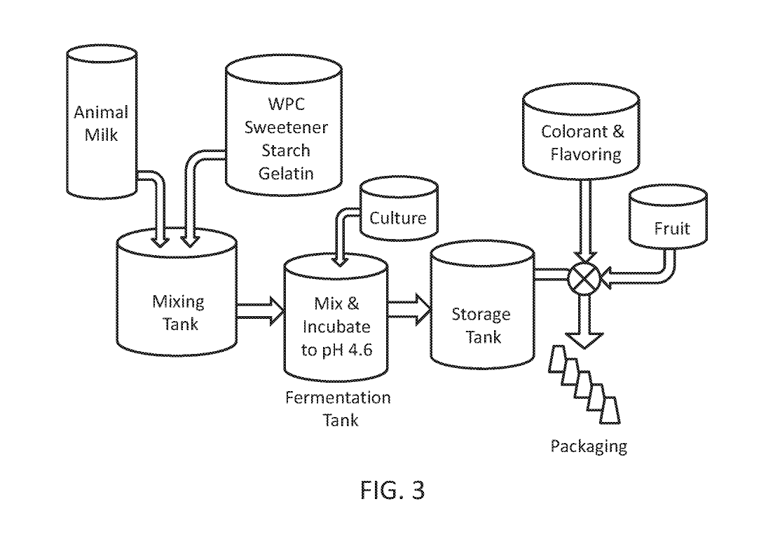

FIG. 3 depicts a typical manufacturing process for fermenting milk to produce dairy-based yogurt. The basic yogurt manufacturing processes generally uses a dairy medium such as milk or a milk component as starting material in a manufacturing process. Various thickening agents/stabilizers (e.g., hydrocolloids such as starches or gelatins), and/or whey protein concentrates are often added to adjust gel structure and/or consistency and the mixture is then heated to allow pasteurization and thickening. To this mixture is added yogurt-producing bacterial culture(s), and fermenting proceeds under heated conditions until the mixture reaches the required level of acidity to produce the yogurt.

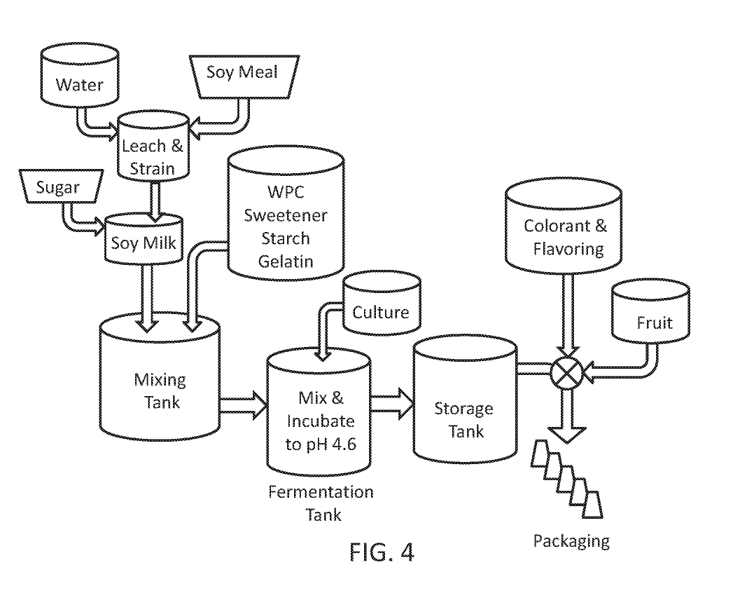

FIG. 4 depicts a typical manufacturing process for fermenting a plant substrate to produce non-dairy yogurt. As shown, non-dairy yogurt can be prepared by leaching soybean meal with an aqueous solution having a pH of 4 to 5 to remove sugars without removing protein, and then leaching a resultant residual sugar-free cake with an aqueous solution having a pH above 7 to dissolve protein material. The solution is strained to remove residual solids. Sugar is added to the filtrate and homogenized to produce a soymilk. The sugar used in the homogenization step is needed to enable f.ermentation. The soy milk is sterilized at about 116° C., and fermented with a lactic culture to produce a non-dairy yogurt.

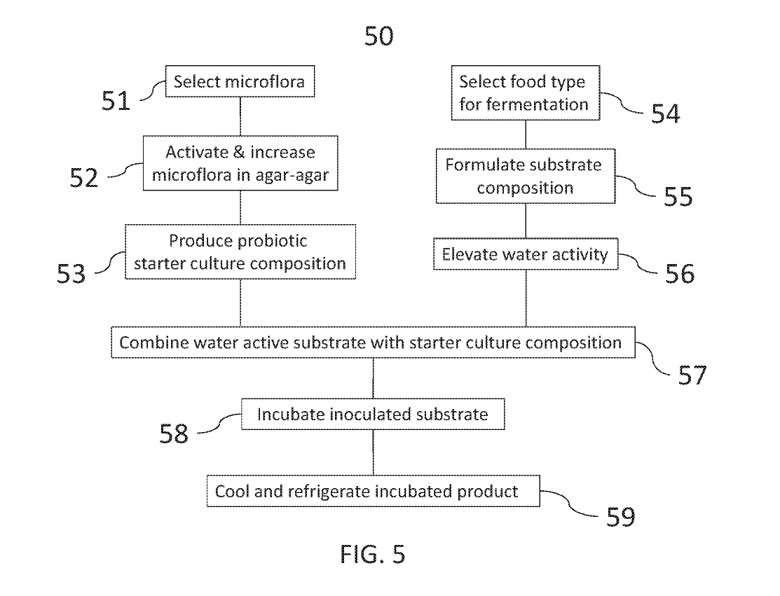

FIG. 5 presents steps in the method provided by the present invention producing a fermented plant product (e.g., vegetable, fruit, legumes) using at least one species of beneficial microllora activated in agar-agar and absent addition of dry salt or brine. Fermentation of plant substrates without salt or brine often lacks a sufficient rate of colonization, resulting in incomplete fermentation and spoiled product. The method of the present invention enables significant acceleration colonization of beneficial microffora, which rapidly produces an acidic environment unsuitable for the growth of spoilage organisms and assures completion of fermentation.

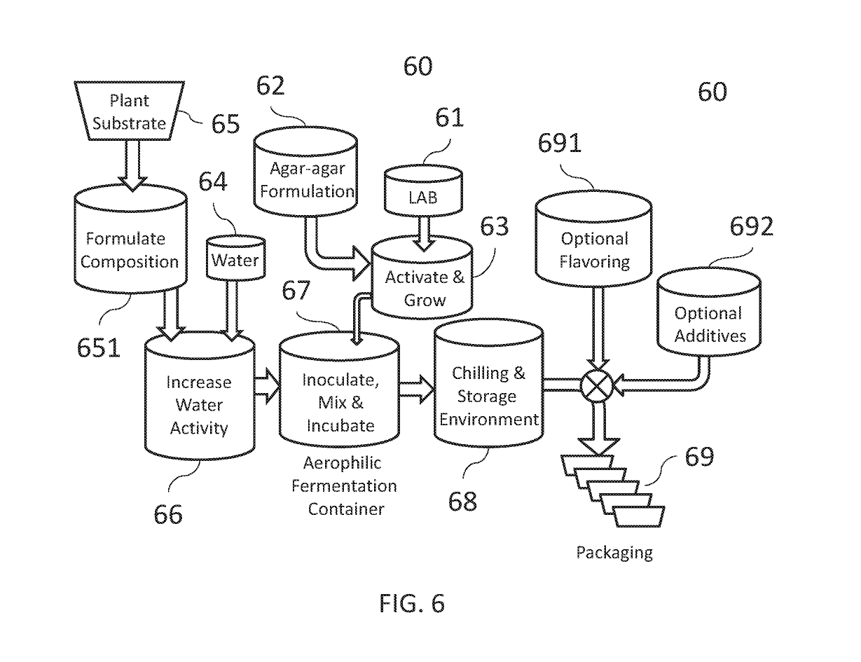

FIG. 6 depicts the process of the present invention providing a method of producing a fermented product derived from plants (e.g., vegetables, grain, fruit or legumes) with beneficial micro-flora activated in agar-agar. The present invention provides formulation and process for making fermented probiotic food products from any of a plurality of plant substrates, without the use of added salt, by increasing the water activity of a plant substrate and accelerating beneficial microffora colonization of the substrate through addition of a starter culture composition comprising at least lactic acid bacteria activated in an agar-agar formulation. Substrate formulation and physical composition may be optimized (e.g. by grinding or shredding) to enable product characteristics closer to traditional artisan flavors and consistencies.

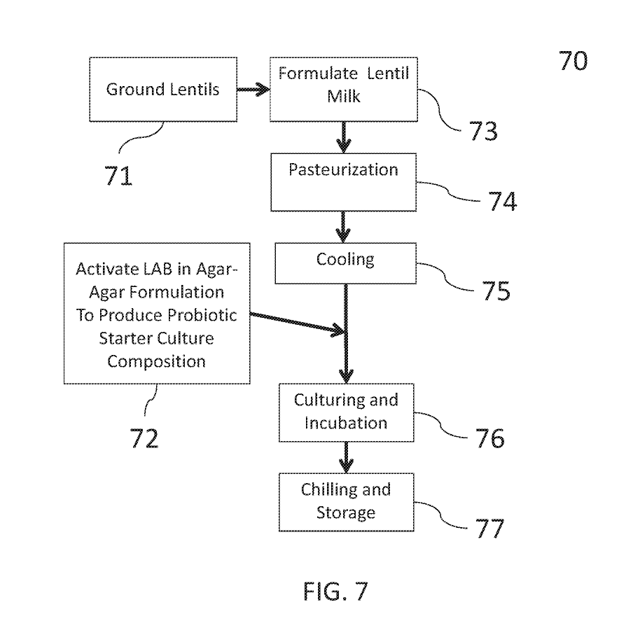

FIG. 7 presents the steps in the process of the present invention producing non-dairy yogurt or kefir. The formulation and process of the present invention provides a fermentation method to manufacture lentil based probiotic yogurt or kefir as a functional food. The method relies on activation of probiotic bacteria in an agar-agar formulation to produce a starter culture composition used to inoculate a lentil milk substrate formed by substantially increasing water activity of ground lentils, and then culturing and incubation of the lentil milk to produce an acidic food product.

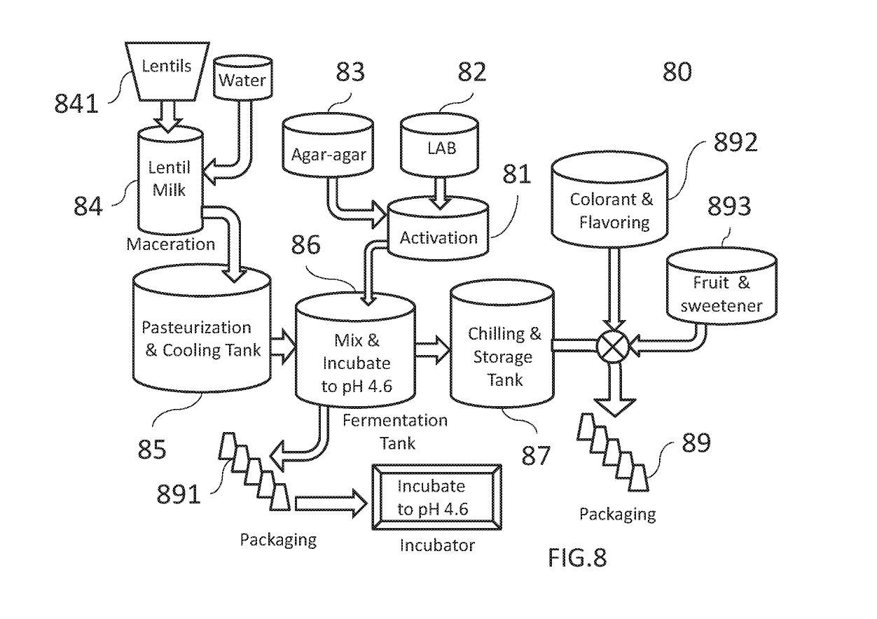

FIG. 8 depicts the process of the present invention providing a method of producing a yogurt-like product derived from lentils and beneficial microffora activated in an agar-agar formulation. The water activity of lentils is increased by creating a lentil-milk comprising at least ground lentils and water. A starter culture composition comprising at least lactic acid bacteria activated in an agar-agar formulation is used to accelerate microflora colonization of the lentil-milk substrate. The substrate formulation and physical composition may be optimized (e.g. by thickening) to enable product characteristics closer to traditional artisan flavors and consistencies. The process and formulation used to produce a yogurt-like product may be adapted to produce a kefir-like product by optimization of the lentil milk substrate and use of ketir grains and alternative microflora.

The method provided by the process of the present invention produces fermented foods of consistent quality, using starter culture compositions comprising at least lactic acid bacteria. (LAB) activated in an agar-agar formulation prior to mixing with a plant substrate. These agar-agar derived starter culture compositions ensure consistency in scaled-up production and accelerate the fermentation process enabling relevant microflora to rapidly colonize the food substrate. The activated LAB produce an acidic state in the starter culture compositions prior to mixing, inhibiting growth of undesirable micro-organisms. Any of the major ENB listed in Table 1, as well as other beneficial microflora, may be used in the method provided by the process of the present invention to produce fermented food products for human consumption, as well as fermented animal feed stocks.

TABLE 1

| Homofermenter | Facultative homofermenter | Obligate heterofermenter |
|---|---|---|
| Enterococcus faecium | Lactobacillus bavaricus | Lactobacillus brevis |
| Enterococcus faecalis | Lactobacillus casei | Lactobacillus buchneri |
| Lactobacillus acidophilus | Lactobacillus coryniformis | Lactobacillus cellobiosus |
| Lactobacillus lactis | Lactobacillus curvatus | Lactobacillus confusus |
| Lactobacillus delbrueckii | Lactobacillus plantarum | Lactobacillus coprophilus |
| Lactobacillusleichmannii | Lactobacillus sake | Lactobacillus fermentatum |
| Lactobacillus salivarius | | Lactobacillus sanfrancisco |
| Streptococcus bovis | | Leuconostoc dextranicum |
| Streptococcus thermophilus | | Leuconostoc mesenteroides |
| Pediococcus acidilactici | | Leuconostoc paramesenteroides |
| Pedicoccus damnosus | | |
| Pediococcus pentocacus | | |

In detail: Referring now to FIG. 5, the process of the present invention 50 may be used to produce a fermented food product from any plant substrate including at least vegetable substrates, fruit substrates, grain substrates, and legume substrates. Whether in liquid or relatively solid form. The formulation and process of the present invention 50 provides a method to produce probiotic functional foods from plant stibstrates. The method steps comprise activation 52 of probiotic bacteria selected 51 from microflora including tactic acid bacteria (LAB) species such as those listed in Table 1. The selected LAB are activated 52 in agar-agar base formulation to produce a starter culture composition 53. A food type (e.g., vegetable, grain, fruit or legume) is selected 54 for f.ermentation and the physical characteristics are amended to formulate 55 a plant substrate composition needed to produce the fermented food product desired. The water activity of the plant substrate composition is elevated 56 by increasing unbound water, which may be accomplished by at least adding water or a complementing plant juice. A complementing juice may be a derivative of the plant type selected for fermentation or an alternative plant type. The water active, plant substrate composition is combined (inoculated) 57 with the starter culture composition 53 comprising activated probiotic bacteria in an agar-agar formulation. The inoculated plant substrate is incubated 58 until the level of acidity reaches the pH range of 3 to 5 and preferably pH 4.6 or below and fermentation is complete. The fermented food product is then allowed to cool 59 and is subsequently refrigerated to slow or substantially stop further fermentation.

The process 50 does not rely on dry-salting or the use of brines to produce finished products. Use of a starter culture composition 53 comprising LAB activated in an agar-agar formulation prior to mixing 57 with a plant substrate eliminates the need for salt in the fermentation process by accelerating growth of beneficial microftora to rapidly colonize the food substrate. This is a critical aspect of the method provided by the process of the present invention 50 enabling fermentation in larger batch sizes without the prior addition of salt. Absent rapid colonization by beneficial microflora, fermentation 58 without addition of salt or brine may be incomplete and the food product may spoil. The process of the present invention 50 provides an effective method to accelerate growth of beneficial microflora enabling production of low sodium fermented food products, as well as fermented animal feed stocks at commercial scale.

Referring now to FIG. 6, the process of the present invention 60 (designated 50 in FIG. 5) provides a method to manufacture at scale probiotic functional foods from at least vegetable, grain, fruit and legume substrates, the method comprising:
a. Activation of lactic acid bacteria (LAB) 61 in an agar-agar formulation 62 to produce
a probiotic starter culture composition 63;
b. Formulation 651 of plant (e.g., vegetable, grain or fruit) substrates 65 assuring elevated water activity 66 by adding unbound water 64 or juice extracted from the same or complementary plants;
c. Culturing and incubation 67 of water active substrates in an aerophilic fermenta ion environment after mixing with the starter composition 63 containing activated probiotic LAB ;
d. Cooling and refrigeration 68 of the incubated fermentation product;
e. Packaging 69 the fermentation product.

The addition of various flavorings 691 such as natural spices or other seasonings may be accomplished after fermentation or for some types of herbal seasonings before fermentation. Other additives 692 such as colorants may also be included in the fermented product.

The formulation and process of the present invention 60 produces a fermented functional food product without addition of salt. This is accomplished by elevating water activity 66 in the food substrate, followed by inoculation 67 with lactic acid bacteria starter culture composition 63 activated in an agar-agar formulation 62 prior to mixing 67 with the substrate 63. The agar-agar formulation 62 may be constructed by dissolving agar-agar (e.g., powder, chips) in water heated to a temperature generally above 80 degrees Fahrenheit (° F). Water at this temperature is sufficient to completely dissolve the agar-agar so it will set up smooth and mix 67 into the p ant substrate composition completely. The agar-agar formulation 62 is allowed to cool below 110° F. before combining 63 with the probiotic bacteria (LAB) 61. Temperatures in the range of 100° F to 105° F. will activate the LAB without killing it. The starter culture composition 63 produced using the formulation and process of the present invention 60 may be utilized in a liquid or a more solidified state depending on the substrate composition. The food substrate formulation 651 may be exhibit any physical characteristic required for a desired fermented product, including but not limited to whole, shredded, ground, crushed or liquefied. Fermentation temperature should be set and maintained at substantially 110° F for 4 to 8 hours depending on the plant substrate 65 selected.

The starter culture composition 63 produced using the process of the present invention 60 may be formulated to exhibit a gelled consistency, which enables the starter culture composition 63 to be molded into and sustain specific shapes, packaged and stored in refrigeration prior to use. Thereafter, the starter culture composition 63 in molded form may be added to any water active vegetable, grain or fruit substrate 66 exhibiting liquid or more solid characteristics to produce a probiotic functional food. The starter culture 63 may also be added to water to produce a probiotic functional beverage, where the starter culture composition 63 may or may not be combined with flavorings 651 or other additives.

Referring now to FIG. 7, the process of the present invention 70 (designated as 50 in FIG. 5) may be used to produce a non-dairy product from a legume substrate comprising ground lentils 71 that exhibits substantially the characteristics of traditional dairy-based yogurt. The process of the present invention 70 may also be used to produce a non-dairy product from a legume substrate comprising ground lentils 71 that exhibits substantially the characteristics of traditional dairy-based kefir. The ingredients in the formulation of the non-dairy product produced by the process of the present invention 70 have low to no known allergies When produced using lentils, which is an essential characteristic of the yogurt product and the kefir product.

The formulation and process of the present invention 70 provides a method to manufacture lentil based probiotic yogurt or kefir as a functional food, the method comprising:
a. Activation 72 of lactic acid bacteria (LAB) in an agar-agar formulation to produce a probiotic starter culture composition;
b. Formulation of lentil milk substrate 73 by increasing water activity of ground lentils and achieving a consistency suitable for producing one of yogurt or kefir;
c. Pasteurization 74 of the lentil milk followed by cooling 75;
d. Culturing and incubation 76 of lentil milk with the probiotic starter culture composition;
e. Cooling and Refrigeration 77 of the fermented product.

Unlike non-dairy based yogurts and kefir produced using alternative substrates, the /products produced using the present invention 70 may include nutrient dense lentils and probiotic microflora activated in an agar-agar formulation 72. These ingredients are superior sources of fiber and protein, exhibit low fat and high omega properties and provide probiotics to the population that cannot consume nuts or soy. A desired product consistency can be achieved by variation of water activity of the ground lentils when formulating 73 the lentil milk. This can be accomplished without straining as is typical in commercial yogurt production processes: straining reduces the nutrient content. Unlike current commercial practice, this new process 70 for making non-dairy yogurt and kefir retains substantially all of the fiber in the product produced, along with the protein. The product does not need gums or other thickeners for the process to produce the desired consistency and texture. According to the method of the present invention 70 applied to fermentation of lentils, it was unexpectedly found through experimentation that anon-dairy yogurt is achieved, activating the probiotic bacteria in an agar-agar formulation 72 instead of a milk medium or by adding sugar to the non-dairy base material; which yogurt has an excellent texture, superior nutrient and fiber composition, and exhibits characteristics similar to dairy-based yogurts of various consistencies.

Probiotics present in lentils were determined to provide an ideal matrix for probiotic growth and produce a nutrient dense probiotic yogurt or kefir product using the process 70 of the present invention. Raffinose oligosaccharides are predominant prebiotics in legumes including lentils. The lentil (Lens culinaris) is an edible legume having about 30% of their calories from protein: the third-highest level of protein, by weight, of any legume or nut, after soybeans and hemp. The proteins in lentils include the essential amino acids isoleucine and lysine. Lentils are deficient in two essential amino acids, methionine and cysteine, however these amino acids can be added to the Lentil Milk 73 or included in the starter culture composition 72. Lentils also contain dietary fiber, folate, vitamin and minerals. Red (or pink) lentils contain a tower concentration of fiber than green lentils (11% rather than 31%). The low levels of Readily Digestible Starch (RDS) 5%, and high levels of Slowly Digested Starch (SDS) 30%, make lentils of great interest to people with diabetes. The remaining 65% of the starch is a resistant starch that is classified RS1, being a high quality resistant starch, which is 32% amylose. Lentils are a good source of iron, having over half of a person's daily iron allowance in a one cup serving.

Experimental Formulation

The method provided by the process of the present invention was discovered through experimentation directed to producing an non-dairy yogurt from lentils. Various means of initiating fermentation were studied where freeze-dried starter cultures were added to ground lentils mixed with water. Mixing the freeze-dried bacteria with a milk base and added to the lentil-water solution resulted in fermentation and produced a yogurt-like product. However, the product was not “dairy free” and therefore not considered vegan (a desired outcome). Using an agar-agar formulation to activate LAB proved to provide a viable alternative for initiating and sustaining a fermentation process that could be optimized to enable product characteristics closest to traditional dairy yogurt. The most successful experimental formulation produced 675 grams of finished product. The specific type of agar-agar used for the experimental formulation was Eden Foods Agar-agar Sea Vegetable Flakes, comprising Sea Vegetable Getidium amansii and Gracilaria verrucosa. However, agar-agar product can be obtained from a variety of manufacturers. The specific lactic acid bacteria (LAB) used to produce the probiotic starter culture composition (72 in FIG. 7) in the experimental formulation was Lactobacillus bulgaricus, Streptococcus thermophilus, in powder form and frozen. The quantity of 0.003 gram was added after activation in water to 500-550 ml lentil milk. To activate the frozen bacteria it was dissolved in warm water (2-3 T). This is done to bring bacteria at room temperature. If the frozen bacteria powder is added directly into the lentil milk then it will not work. For the experimental non-dairy yogurt formulation, the activation instructions for the specific bacteria provided by the manufacturer were followed before combining the bacteria into the agar-agar formulation. The experimental formulation used Lactobacillus bulgaricus, Streptococcus thermophilus which are the typical yogurt cultures used for making dairy yogurt. These cultures are available from Danisco and these cultures are used in dairy industry to make dairy yogurt. The product number of this Danisco culture is yo-mix 495 LYO 250 DCU. The following comprise ingredients used in the experimental formulation:

⅓ cup White lentils rinsed

2 cups water

2 tewoons agar-agar

½ cup water

0.003 grams freeze dried probiotic bacteria

The Wowing equipment was used in the food laboratory to produce various experimental compositions of a lentil based yogurt-like product:

| | | |
|---|---|---|
| Thermometer | 2 Glass bowls | 4 quart pot |
| 1-8 Oz. bowl | High powered blender | Microwave |
| Aluminum foil | Rubber spatula | Incubator |

Referring now to FIG. 8, the process of the present invention 80 (50 in FIG. 5) provides a method to manufacture at scale lentil based probiotic yogurt as a functional food using substantially of the components of lentil seeds. The method was determined and validated through experimentation. A functional food is a. food given an additional function (often one related to health-promotion or disease prevention) by adding new ingredients or more of existing ingredients. A functional food is a natural or processed food (including in general both in solid and liquid form) that contains known biologically-active compounds which when in defined quantitative and qualitative amounts provides a clinically proven and documented health benefit. In addition to probiotic benefits, fermentation of the lentil seeds reduces their anti-nutrient properties making the nutritional components more available for digestion. The method provided by the process of the present invention involves the following steps:
A. A.ctivation of LAB 82 in an agar-agar formulation 83 to produce a probiotic starter culture composition 81;
B. Formulation of Lentil Milk 84 by elevating the water activity of ground lentils 841 followed by pasteurization and cooling 85;
C. Culturing and incubation 86 of Lentil Milk combined with the starter culture composition 81;
D. Cooling and Refrigeration 87

A. Activation of LAB in Agar-Agar to Produce Probiotic Starter Culture

Frozen lactic acid bacteria 82 in powder form (freeze dried) was used in producing 81 the probiotic starter culture composition. The LAB 82 were added to water heated to approximately 100° F. This is done to wake up (activate) the bacteria getting them ready to feed and grow. It is important to use water in the range of 98° F to 105° F. and preferably at 100° F. degrees because it was found to be the optimal temperature to activate LAB from the frozen state.

The agar-agar formulation 83 was constructed by heating water heated to a temperature generally above 180° F then mixing in agar-agar and agitating to completely dissolve the agar-agar. It was found that maintaining the agar-agar formulation in a relatively liquid state was necessary so it would mix into the lentil milk 84 completely. Agar-agar behaves very similar to gelatin so it thickens as it cools. The agar-agar formulation was cooled to a temperature below 110° F. and then the probiotic bacteria were added and allowed to activate 81. the bacteria are put in the agar-agar formulation before it has cooled to the optimum temperature for bacterial growth (e.g. 110° F.) there is risk of destroying the bacteria. Do not allow the agar-agar formulation to solidify or it won't mix well later in the process. The agar-agar formulation 83 needs to stay in liquid form. If it is allowed to solidify before it is mixed 86 into the lentil milk 84 it wilt not disperse and it was found to create a lumpy yogurt or kefir. Agar-agar is a food-grade gelatin made from seaweed that was determined through experimentation to provide a satisfactory substitute to a milk or sugar medium in fermentation. A agar-agar formulation comprising at least agar-agar and water is used in the process of the present invention 80 to jump start (accelerate) growth of the bacteria. The bacteria are added directly 81 to the agar-agar formulation once the solution has reached the optimal temperature at or below 110° F. Other beneficial ingredients such as but not limited to herbs may be included in the agar-agar formulation.

B. Formulation of Lentil

The lentils 841 were subjected to a thorough (e.g., 3 times) rinse in water. This was considered necessary to assure removal of any foreign objects that may have been present before maceration or grinding. The following rinse method was found effective during experimentation. immerse the lentils 841 in water and stir the lentils for 20 seconds, then remove anything that floats or is discolored. Then drain off the water and repeat this process 2 more times. This method was also found effective to leech out extra starches and bitterness that reside in the husk of the lentils. The extra starch needs to be removed because it may create an undesirable aftertaste and thickness in the yogurt or kefir.

The rinsed lentils were macerated in water using a high speed blending process for at least three (3) minutes to produce Lentil 84 was found that the blending process needs to run for at least three (3) minutes to puree the lentils into smooth milk. If it is not blended enough the yogurt or kefir was grainy and contained chunks of hard lentils. Elevating water activity of the ground lentils was essential. Test revealed that using a 6 to 1 volumetric ratio of water to lentils produced the correct thickness for yogurt during the heating stage. A greater volume of water may be used in producing drinkable yogurt or kefir. Variation of the ratio can be used to alter the viscosity and texture of the finished product, where a more liquid consistency is desired. An alternative to maceration or grinding of whole lentils is substitution of lentil flour, which was used in the later stages of testing. Lentil flour is widely available from suppliers in Canada such as Northern uitma Corp.

The milk 84 was transferred from the blending process to a pasteurization process 85, and slowly heated to at least 165° F. The elevated temperature was sustained for at least 15 seconds, with the temperature being verified using a thermometer. The standards for cooking and reheating foods set by the U.S. Food & Drug Administration (FDA) and Servsafe certification is to bring foods to 165° F. This is done to assure the levels of potentially harmful bacteria are brought down to a safe level. It also creates the optimal thickness from the natural starches and proteins in the lentil milk 84 as they firm up when heating during Pasteurization 85. During heating 85, the lentil milk 84 requires constantly stirring or agitation so the starches from the lentils don't build up on the bottom of the Pasteurization container 85 and the product will remain smooth. It is critical that a minimum of 165° is reached so harmful bacteria are not present to contaminate the lentil-based yogurt or kefir product during fermentation 86. The process of the present invention 80 meets FDA standards for pasteurization.

The heat source was removed from the lentil milk in the pasteurization container 85 and the mixture allowed to cool to a temperature below 110° F. Experimentation has shown that temperature in the range of 100° F to 110° F. and preferably at 110° F. is the optimal growth temperature for the probiotic bacteria. If the bacteria are mixed in while the lentil milk is too hot (substantially above 110° F) the fragile bacteria may be destroyed and rendered ineffictive.

C. Culturing and Incubation of Lentil Milk

The lentil milk 84 was combined 86 after Pasteurization 85 with the probiotic starter culture composition 81 in a closed container for fermentation with the temperature set and maintained at 110° F for substantially 8 hours. A temperature of 110° F was found to be the optimal for the growth of the probiotic bacteria in the lentil milk 84. A fermentation time of 8 hours was found most suitable for the pH to drop to the desired level between pH 4 and pH 5 and preferably pH 4.6 for the taste profile comparable to traditional dairy yogurt and kefir.

D. Cooling and Refrigeration

The lentil-based yogurt was removed from the fermentation container 86 and cooled to a temperature of 36° F. a chilling container 87. Once the yogurt (or kefir) reaches the desired pH of 4.6 the growth of the bacteria needs to be stopped by cooling. Bringing the temperature down below 4 degrees Centigrade (° C.) will nearly stop the growth of the bacteria in the fermented product.

Referring to FIG. 8, depending on the type of yogurt, the incubation process is done either in a large tank of several hundred gallons or in the final individual containers. Stirred yogurt is fermented 86 in bulk and then poured into the final selling containers 89. Set yogurt, also known as French style, is allowed to ferment right in the container 891 it is sold in in both instances, the lactic acid level is used to determine when the yogurt is ready. In acid level may be determined by taking a sample of the product and titrating it with sodium hydroxide. A value of at least 0.9% acidity and pH of about 4.4 to 4.6 are the current minimum standards for yogurt manufacture the United States. When the yogurt reaches the desired acid level, and it has cooled, if fermented in bulk it may be modified as necessary and dispensed into containers.

In contrast to production processes used to produce soy-based yogurt as shown in FIG. 4, the method provided by the process of the present invention illustrated in FIG. 8 requires no added sugar to make the lentil-based yogurt (or a kefir) product. Soy yogurt is made using soy milk, adding yogurt bacteria (Lactobacillus delbrueckii subsp. Bulgaricus and Streptococcus salivarius subsp. thermophilus), adding sweeteners such as fructose, glucose, or sugar. Sugar is added to unsweetened soy milk to promote bacterial fermentation. Soy milk on its own lacks the lactose (milk sugar) that is the basic food for the yogurt bacteria. Instead, an agar-agar formulation 83 is used in the method provided by the process of the present invention 80 to initiate and sustain fermentation 86 of the water active lentil milk 84. In addition, the processes used to make soy-based yogurt requires straining the soy milk to remove residual solids. Straining the mixture removes both fiber and nutrients. The method provided by the process of the present invention 80 retains substantially all of the lentil fiber and nutrients, as no straining of the lentil milk 84 is needed.

Similarly, coconut milk yogurt is made using essentially the same process used to produce both soy-based yogurt (FIG. 4) and dairy yogurt (FIG. 3). Straining is used to remove residual solids, and thickeners are usually added. While coconut milk yogurt has natural sugars that can promote fermentation by bacteria, and it can have a similar consistency and flavor as dairy-based yogurt, it lacks any substantial amount of protein and fiber.

Mild yogurt cultures, which are preferred in the process of the present invention 80 for quality reasons, require fermentation in the range of 6 to 12 hours. The yogurt culture may contain any bacteria known in the art (see Table 1) to be useful for dairy product fermentation, but Streptococcus thermophilus and Lactobacillus billgaricus are preferred. The present method digresses from conventional methods as described above, however, it can be used to produce any form of yogurt including a gel-like form, stirred yogurt, and drinking yogurt in a liquid form.

Several varieties of probiotic bacteria commonly found in kefir products e Table I), but not commonly found in yogurt are anticipated for use in the process of the present invention 80 to produce a kefir-like product. These probiotic bacteria may include Lactobacillus acidophilus, Bifidobacterium fidum, Streptococcus thermophilus, Lactobacillus delbrueckii subsp. bulgaricus, Lactobacillus helvelicus, Lactobacillus kefiranofaciens, Lactococcus lactis, and Leuconostoc species. Use of beneficial yeasts is also anticipated, such as Saccharomyces kefir and Torula kefir. The viscosity of the finish product may be controlled by adjusting the composition ratio of the lentil milk 84 from the volumetric optimum of 6 to 1 water to lentils required for a yogurt-like product.

Other components, such as flavoring & coloring or 892 fruit & sweetener 893 including artificial sweeteners, can optionally be added prior to storage, during storage, or between storage and packaging. The fruit preparations can be fruit syrup, jams, marmalades, fruit preserves, fruit jelly, fruit sweetened fruit pulp, fruit concentrate, frozen fruits, and can include sugar, natural flavors, and colorants. The fruit preparation 893 can be added before filling the yogurt into the packaging, forming a visible deposit on the bottom, or the preparation can be added on top of the yogurt or can be stirred into the yogurt in a storage or process tank. Natural or synthetic sugars such as fructose, dextrose, corn syrup solids, lactose, aspartame, and sucrose may be used. Such sugars may be employed singly or in combination. Moreover, artificial sweeteners such as, for example, edible saccharin salts, dipeptide salts and the like may be used. The additives can be added before or after rapid cooling of the yogurt composition.

In addition to the above additives, a yogurt or kefir preparation produced using the present invention 80 may include a wide variety of other additives. These additives include buffering agents, vitamins, minerals, appetite suppressants, preservatives, and the like. These additives, while not necessary, should only be present in amounts no as not to adversely affect the overall taste, appearance, and acceptability of the final yogurt food product.

The yogurt and kefir products produced using the process of the present invention 80 may be preserved by, thr example, chemical or thermal preservation and by aseptic production methods. Chemical preservation may be accomplished by using preservatives such as sorbic acid to prevent growth of harmful yeasts and molds. Thermal preservation may be accomplished by storing the yogurt at temperatures that prevent the growth of harmful microorganisms.

The starter culture composition produced using the method provided by the process of the present invention may be used to initiate and accelerate fermentation of seed grains used in animal feed stocks without addition of salt or brine. Use of the starter culture composition of the present invention assures rapid colonization in a feed stock substrate by beneficial micro-flora. Rapid colonization in animal feed stocks serves to prevent spoilage in low temperature environments typical of farms due to growth of yeasts or other undesirable microflora. Fermentation of seed grains reduces their anti-nutrient properties, making seed grain feed stocks more available for digestion.

The skilled artisan will appreciate that the present invention is suitable for use in producing a variety of fermented plant-based products, and is not limited by the specific examples cited herein. While particular emphasis has been directed towards experimental results obtained by fermenting water active ground lentils (e.g., lentil milk) to produce products such as yogurt and. kefir, the skilled artisan will also appreciate that the present invention is also suitable for use in fermenting any type of plant substrate to produce functional foods for both humans and animals. Further, the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A process providing a method for production of fermented probiotic functional foods from plants, the method comprising:
activation of lactic acid bacteria in an agar-agar formulation to produce a probiotic starter culture composition;
formulation of plant substrates assuring elevated water activity hospitable to fermentation;
culturing and incubation of said plant substrates with activated lactic acid bacteria in said probiotic starter culture composition acidifying said plant substrate to within a specific pH range;
cooling said plant substrate, and
refrigerating said plant substrate to retard further fermentation,
wherein said plant substrate comprises or is derived from at least one of a vegetable, grain, fruit and legume edible by humans or animals.

2. The process of claim 1, wherein fermentation proceeds absent addition of salt or brine.

3. The process of claim 1, wherein said starter culture composition comprises an agar-agar formulation including at least one lactic acid bacteria species within at least one of the Lactobacillus and Bilidobacterium bacterial genera.

4. The process of claim 3, wherein said starter culture composition exhib e of a liquid or a more solidified state.

5. The process of claim 4, wherein said starter culture composition exhibits a gelled consistency molded into one or a plurality of shapes.

6. The process of claim 5, wherein said starter culture composition is packaged and preserved for subsequent addition to any water active vegetable or fruit substrate exhibiting liquid or more solid characteristics to produce a probiotic functional food.

7. The process of claim 6, wherein said starter culture composition includes any one or combination of lactic acid bacteria strains useable to produce fermented vegetable and fruit products.

8. The process of claim 3, wherein said lactic acid bacteria comprise at least one of Streptococcus thermaphilus and Lactobacillus bulgaricus.

9. The process of claim 3, wherein said lactic acid bacteria comprise at least one of Lactobacillus acidophilus, Bilidobacterium bifidurn, Streptococcus thermophilus, Lactobacillus delbrueckii subsp. bulgaricus, Lactobacillus helveticus, Lactobacillus kefiranafaciens, Lactococcus lactis, and Leuconostoc species.

10. The process of claim 1, further comprising construction said agar-agar formulation by heating to a temperature above 165 degrees Fahrenheit a solution of water mixed with agar-agar, where the agar-agar is completely dissolved.

11. The process of claim 10, further comprising cooling said agar-agar formulation to a temperature in the range of 105 degrees Fahrenheit to about 110 degrees Fahrenheit.

12. The process of claim 11, further comprising adding said probiotic bacterial culture to said agar-agar formulation.

13. The process of claim 12, further comprising maintaining said agar-agar formulation in a liquid state.

14. The process of claim 12, further comprising transitioning said agar-agar formulation to a more solidified state.

15. The process of claim 14, further comprising transitioning said agar-agar formulation to a moldable gel-like consistency.

16. The process of claim 11, further comprising altering the physical characteristics of said plant substrate to enhance water activity and support fermentation.

17. The process of claim 16, wherein said alteration of physical acteri sties comprises one of grinding, shredding, crushing or liquefying said plant substrate.

18. The process of claim 16, wherein said alteration of physical characteristics comprises softening or heating said plant substrate.

19. The process of claim 1, wherein said plant substrate comprises culinary vegetables and said fruit substrates comprise botanical fruits.

20. The process of claim 1, wherein said plant substrates comprise seed grains for animal feed stocks.

21. A process providing a method for production of a lentii-based probiotic food product exhibiting characteristics similar to dairy-based yogurt, said method comprising:
activation of probiotic bacteria in an agar-agar formulation to produce a starter ciuture composition for initiating fermentation;
formulation of lentil milk combining at least water and ground lentils or lentil flour;
culturing and incubalentil milk combined with said starter culture composition to reach a desired pH,
halting fermentation by cooling and refrigeration.

22. The process of claim 21, further comprising constructing said agar-agar formulation by heating at least a solution of water and agar-agar above 165 degrees Fahrenheit, where the agar-agar is completely dissolved.

23. The process of claim 22, further comprising cooling said agar-agar formulation to a perature in the range of 105 degrees Fahrenheit to about 110 degrees Fahrenheit.

24. The process of claim 23, further comprising adding said probiotic bacteria to said agar-agar formulation to produce a starter culture composition.

25. The process of claim 24, further comprising maintaining said agar-agar formulation in a liquid state.

26. The process of claim 25, further comprising rinsing said lentils to substantially remove any foreign objects.

27. The process of claim 26, further comprising submerging said lentils in water and separating flotsam.

28. The process of claim 27, further comprising draining said rinse water and submerging said lentils at least two more times to substantially leech out extra starches and bitterness that reside in the husk of said lentils.

29. The process of claim 28, further comprising macerating lentils in water in a volumetric ratio of 6 parts water to 1 part lentils at least three minutes to puree the lentils into smooth lentil milk.

30. The process of claim 29, further comprising slowly heating said lentil milk to 165 degrees Fahrenheit for at least 15 seconds to bring the levels of potentially harmful bacteria down to a safe level and create the optimal thickness from the natural starches and proteins as they firm up while heating.

31. The process of claim 30, further comprising constantly stirring or agitating said lentil milk so that the product will remain smooth.

32. The process of claim 31, further comprising allowing said lentil milk to cool to 110 degrees Fahrenheit.

33. The process of claim 32, further comprising combining said starter culture composition with said lentil milk, and fermenting at substantiallyl10 degrees Fahrenheit for 6 to 8 hours until the pH of the mixture reaches a desired level

34. The process of claim 33, further comprising cooling said starter culture composition and said lentil milk to a temperature below 40 degrees Fahrenheit, and preferably below 36 degrees Fahrenheit to substantially stop the growth of bacteria.

35. A process providing a method for production of a lentil-based probiotic food product exhibiting characteristics similar to dairy-based yogurt, said method comprising:
activation of probiotic bacteria. in an agar-agar tormulation to produce a starter culture composition;
formulation of a substantially smooth lentil milk combining macerated lentils or lentil flour with water in a volumetric ratio of 6 parts water to 1 part lentils;
pasteurizing said lentil milk by slowly heating said lentil milk to 165 degrees Fahrenheit for at least 15 seconds;
culturing and incubation of said lentil milk allowing said lentil milk to cool to 110 degrees Fahrenheit then combining said starter culture composition with said lentil milk, and fermenting at substantiallyl10 degrees Fahrenheit for 6 to 8 hours until the pH of the mixture reaches a desired level;
cooling after incubation said combined starter culture composition and lentil milk to a temperature below 40 degrees Fahrenheit, and preferably below 36 degrees Fahrenheit to nearly stop the growth of bacteria.

36. The process of claim 35, wherein said agar-agar formulation is brought from a temperature above 165 degrees Fahrenheit to a temperature in the range of 105 degrees Fahrenheit to about 110 degrees Fahrenheit.

37. The process of claim 35, further comprising submerging said lentils in water, draining said water and submerging said lentils at least two more times to substantially leech out extra starches and bitterness that reside in the husk of said lentils.

38. A process providing a method for production of a lentil-based probiotic food product exhibiting characteristics similar to kefir, said method comprising:
activation of probiotic bacteria in an agar-agar formulation to produce a starter culture composition for initiating fermentation;
formulation of lentil milk combining at least water and ground lentils or lentil flour;
including beneficial yeasts;
culturing and incubation of said lentil milk combined with said starter cuituie composition to reach a desired pti,
halting fermentation by cooling and refrigeration.

39. The process of claim 39, wherein said probiotic bacteria is selected from the group comprising Lactobacillus acidophilus, Bifidobacterium bifidum, Streptococcus thermophilus, Lactobacillus delbrueckii subsp. bulgaricus, Lactobacillus helveticus, Lactobacillus kefiranofaciens, Lactococcus lactis, and Leuconostoc species.

40. The process of claim 39, wherein said beneficial yeasts include at least one of Saccharomyces kefir and Torula kefir.

41. A probiotic starter culture composition for accelerating fermentation processes, the composition comprising:
at least one species of lactic acid bacteria activated in a solution of agar-agar dissolved in water, said composition exhibiting a gel-like consistency suitable for molding said composition into a plurality of shapes and sustaining said shapes after molding,
wherein said starter culture composition when added to water active plant substr s initiates and accelerates fermentation of said plant substrate.

42. The probiotic starter culture composition of claim 41, further comprising at least one of a flavoring or colorant.

43. The probiotic starter culture composition of claim 41, wherein said composition is usable in fermentation of plant substrates comprising any one or a plurality of vegetables, fruits, grains, and legumes.

44. The probiotic starter culture composition of claim 41, wherein said composition is usable in fermentation of plant substrates comprising seed grains for animal feed stocks.

45. The probiotic starter culture composition of claim 41, wherein said composition is usable as an additive in water or fruit and vegetable juices to impart probiotic properties.

46. A lentil-based probiotic food product exhibiting characteristics similar to dairy-based yogurt or kefir, said food product produced by a method including the steps:
activation of probiotic bacteria in an agar-agar formulation to produce a starter culture composition;
formulation of lentil milk using one of lentil flour or macerated lentils and water;
culturing and incubation of said lentil milk using said starter culture composition, cooling and refrigeration.
